(19) United States
(12) Patent Application Publication
(10) Pub. No.: US 2009/0209757 A1
(43) Pub. Date: Aug. 20, 2009

(54) Processes for the preparation and purification of paliperidone palmitate

(76) Inventors: Santiago Ini, Haifa (IL); Yaron Shmuely, Hedera (IL); Osnat Porter-Kleks, Petach-Tikva (IL); Kobi Chen, Kfar-Saba (IL); Eli Lancry, Modiin (IL); Claude Singer, Kfar Saba (IL)

Correspondence Address: KENYON & KENYON LLP, ONE BROADWAY, NEW YORK, NY, 10004, US

(21) Appl. No.: 12/319,899

(22) Filed: Jan. 12, 2009

Related U.S. Application Data
(60) Provisional application No. 61/020,222, filed on Jan. 10, 2008.
(60) Provisional application No. 61/037,586, filed on Mar. 18, 2008.
(60) Provisional application No. 61/055,690, filed on May 23, 2008.
(60) Provisional application No. 61/085,503, filed on Aug. 1, 2008.
(60) Provisional application No. 61/091,808, filed on Aug. 26, 2008.

(51) Int. Cl. C07D 471/04; C07C 51/42

(52) U.S. Cl. 544/279; 562/512

(57) ABSTRACT

The present invention encompasses processes for the preparation and purification of paliperidone palmitate.

### CROSS-REFERENCE TO RELATED APPLICATIONS

This application claims the benefit of U.S. Provisional Application Nos. 61/020,222, filed Jan. 10, 2008; 61/037,586, filed Mar. 18, 2008; 61/055,690, filed May 23, 2008; 61/085,503, filed Aug. 1, 2008; and 61/091,808, filed Aug. 26, 2008, the disclosures of which are hereby incorporated by reference.

### FIELD OF INVENTION

The present invention encompasses processes for the preparation and purification of paliperidone palmitate.

### BACKGROUND OF THE INVENTION

Paliperidone, 3-[2-[4-(6-fluorobenzo[d]isoxazol-3-yl)-1-piperidyl]ethyl]-7-hydroxy-4-methyl-1,5-diazabicyclo[4.4.0]deca-3,5-dien-2-one, is a 5-HT antagonist belonging to the chemical class of benzisoxazole derivatives and is a racemic mixture having the following structural formula:

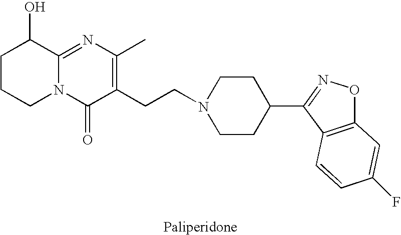

Paliperidone (“PLP”) is a metabolite of risperidone. Marketed under the trade name Invega®, paliperidone is an anti-psychotropic agent approved in the United States for the treatment of schizophrenia.

Processes for the synthesis of paliperidone are purportedly described in U.S. Pat. No. 5,158,952 (“US '952”) and PCT Publication No. WO 96/23784 (“WO '784”). US '952 and WO '784 also describe a process for the synthesis of (3-(2-chloroethyl)-2-methyl-9-benzyloxy-4H-pyrido[1,2-a]-pyrimidine-4-one), a precursor of paliperidone and are hereby incorporated by reference.

The palmitate salt of paliperidone can be chemically named as 3-[2-[4-(6-fluoro-1,2-benzoisoxazol-3-yl)-1-piperidyl]ethyl]-6,7,8,9-tetrahydro-9-hydroxy-2-methyl-4H-pyrido[1,2-a]pyrimidin-4-one palmitate ester and has the following formula:

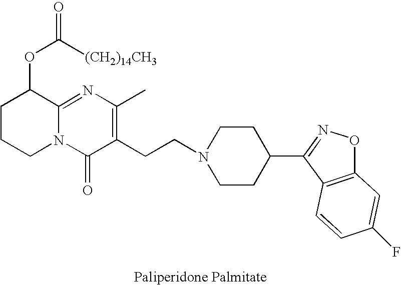

Paliperidone palmitate (“PLP-P”) is described in U.S. Pat. Nos. 5,158,952 and 5,254,556 (“US '556”). US '556 purportedly discloses processes for the preparation of both the decanoyl and the acetyl derivatives of paliperidone palmitate.

Other processes for the preparation of PLP-P are purportedly described in U.S. Pat. No. 6,077,843 (“US '843”) and PCT Publication No. WO 99/25354 (“WO '354”).

Like any synthetic compound, paliperidone palmitate can contain extraneous compounds or impurities. These impurities may include unreacted starting materials, by-products of the reaction, products of side reactions, and/or degradation products.

Impurities in paliperidone palmitate or any active pharmaceutical ingredient (“API”) are undesirable and, in extreme cases, might even be harmful to a patient being treated with a dosage form of the API.

Identifying impurities of an API produced in a manufacturing process is crucial for commercialization. The U.S. Food and Drug Administration (“FDA”) requires that the impurities which arise from the production process be maintained within set limits. For example, in its ICH Q7A guidance for API manufacturers, the FDA specifies the quality of raw materials that may be used and acceptable process conditions, such as temperature, pressure, time, and stoichiometric ratios. The FDA also sets conditions for purification steps, such as crystallization, distillation, and liquid-liquid extraction. See ICH Good Manufacturing Practice Guide for Active Pharmaceutical Ingredients, Q7A, Current Step 4 Version (Nov. 10, 2000).

The product of a chemical reaction is rarely a single compound free of impurities which complies with pharmaceutical standards. Side products and by-products of the reaction and adjunct reagents used in the reaction will, in most cases, also be present in the product. At certain stages during processing of an API, such as paliperidone palmitate, the API must be analyzed for purity, preferably, by using high performance liquid chromatography (“HPLC”) or thin-layer chromatography (“TLC”) to determine if the product is suitable for continued processing and, ultimately, for use in a pharmaceutical product. The FDA requires that an API is as free as possible of impurities, so that the product is as safe as possible for clinical use. For example, the FDA recommends that the amounts of some impurities be limited to less than 0.1 percent. See ICH Good Manufacturing Practice Guide for Active Pharmaceutical Ingredients, Q7A, Current Step 4 Version (Nov. 10, 2000).

The pure API may give rise to thermal behavior different from that of the non-pure API. Thermal behavior originating from melting points, change in weight in relation to change in temperature and phase transition can be measured in the laboratory by such techniques as capillary melting point, thermogravimetric analysis (TGA) and differential scanning calorimetry (DSC). As a result, thermal behavior of a product can be used to distinguish the pure API from non-pure API.

Generally, side products, by-products, and adjunct reagents (collectively “impurities”) are identified spectroscopically, or in combination with another physical method. The identity of the impurity can be associated with a peak position in a chromatogram, or a spot on a TLC plate (Strobel, p. 953, Strobel, H. A.; Heineman, W. R., Chemical Instrumentation: A Systematic Approach, 3rd ed. (Wiley & Sons: New York 1989)). Thereafter, the impurity can be identified, e.g., by its relative position in the chromatogram, where the position in a chromatogram is conventionally measured in minutes between injection of the sample on the column and elution of the particular component through the detector. The relative position in the chromatogram is known as the “retention time.” The retention time varies daily or even over the course of a day based upon the condition of the instrumentation, as well as many other factors. To mitigate the effects such variations have upon accurate identification of an impurity, practitioners use the “relative retention time” (“RRT”) to identify impurities (Strobel, p. 922). The RRT of an impurity is its retention time divided by the retention time of a reference marker. In theory, paliperidone palmitate itself could be used as the reference marker, but as a practical matter it is present in such a large proportion in the mixture that it can saturate the column, leading to irreproducible retention times as the maximum of the peak can wander (Strobel, FIG. 24.8(b), p. 879) illustrating an asymmetric peak observed when a column is overloaded. Thus, it may be advantageous to select a compound other than the API to add to the mixture in a sufficiently large amount to be detectable and a sufficiently low amount as not to saturate the column, and to use that compound as the reference marker.

Those skilled in the art of drug manufacturing research and development understand that a compound in a relatively pure state can be used as a “reference standard.” A reference standard is similar to a reference marker, which is used for qualitative analysis only, but is used to quantify the amount of the compound of the reference standard in an unknown mixture, as well. A reference standard is an “external standard,” when a solution of a known concentration of the reference standard and an unknown mixture are analyzed using the same technique (Strobel, p. 924; Snyder, p. 549, Snyder, L. R.; Kirkland, J. J. Introduction to Modern Liquid Chromatography, 2nd ed. (John Wiley & Sons: New York 1979)). The amount of the compound in the mixture can be determined by comparing the magnitudes of the detector responses. See also U.S. Pat. No. 6,333,198, incorporated herein by reference.

The reference standard can also be used to quantify the amount of another compound in the mixture if a “response factor,” which compensates for differences in the sensitivity of the detector to the two compounds, has been predetermined (Strobel, p. 894). For this purpose, the reference standard is added directly to the mixture and is known as an “internal standard” (Strobel, p. 925; Snyder, p. 552).

The reference standard can even be used as an internal standard when, without the addition of the reference standard, an unknown mixture contains a detectable amount of the reference standard compound using a technique known as “standard addition”. In a “standard addition,” at least two samples are prepared by adding known and differing amounts of the internal standard (Strobel, pp. 391-393; Snyder, pp. 571, 572). The proportion of the detector response due to the reference standard present in the mixture without the addition can be determined by plotting the detector response against the amount of the reference standard added to each of the samples, and extrapolating the plot to zero (See, e.g., Strobel, FIG. 11.4, p. 392).

There is a need in the art for processes for preparing paliperidone palmitate and processes for preparing purified paliperidone palmitate. There is also a need for methods of determining the amount of impurity in paliperidone palmitate comprising performing HPLC analysis with paliperidone myristate or paliperidone stearate as a reference marker and a reference standard.

Further, there is a need in the art for additional processes for the purification of paliperidone palmitate via a simple recovery process.

### SUMMARY OF THE INVENTION

In one embodiment, the invention encompasses 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, sodium salt (“paliperidone alkoxide”) represented by the following formula:

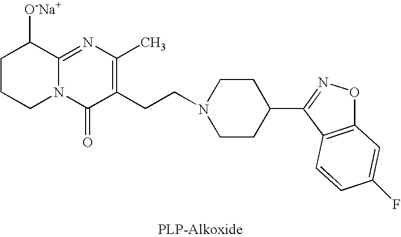

In another embodiment, the invention encompasses a process for preparing paliperidone alkoxide comprising the steps of: a) combining paliperidone with at least one organic solvent to obtain a suspension; b) adding at least one base to the suspension to obtain a reaction mixture; and c) maintaining the mixture for a length of time sufficient to obtain paliperidone alkoxide.

In another embodiment, the present invention encompasses a process for preparing paliperidone palmitate comprising: combining paliperidone alkoxide and palmitoyl chloride to obtain a reaction mixture; and maintaining the mixture for a length of time sufficient to obtain paliperidone palmitate.

In another embodiment, the present invention encompasses a process for preparing paliperidone palmitate comprising combining paliperidone, at least one organic solvent, and at least one base to obtain a reaction mixture, adding palmitoyl chloride to the reaction mixture; and maintaining the mixture for a length of time sufficient to obtain paliperidone palmitate.

In another embodiment, the present invention encompasses paliperidone palmitate containing less than about 0.2% by HPLC of any other ester with a fatty acid.

In another embodiment, the present invention encompasses a process for preparing paliperidone palmitate comprising combining paliperidone, symmetric or asymmetric palmitic anhydride, a pyridine derivative, and an organic solvent to obtain a suspension and maintaining the suspension for a length of time sufficient to obtain paliperidone palmitate.

In another embodiment, the invention encompasses a process for preparing paliperidone palmitate comprising: combining paliperidone, palmitic acid, 4-dimethylamino pyridine (“DMAP”) and an organic solvent to obtain a reaction mixture, adding an acid chloride selected from the group consisting of acetyl chloride, pivaloyl chloride, benzoyl chloride and thionyl chloride to the reaction mixture and maintaining the mixture for a length of time sufficient to obtain paliperidone palmitate.

In another embodiment, the present invention encompasses a process for purifying palmitic acid comprising dissolving palmitic acid in a solvent selected from the group consisting of saturated or unsaturated, linear or branched, cyclic or acyclic C₅ to CB hydrocarbons, C₃ to C₆ ketones, C₆ to C₁₂ aromatic hydrocarbons, C₂ to C₆ alkyl acetates and acetonitrile to obtain a suspension, maintaining the suspension for a length of time sufficient to obtain crystalline palmitic acid.

In one embodiment, the present invention encompasses a process for preparing paliperidone palmitate comprising the steps of: combining paliperidone, palmitic acid, N,N′-dicyclohexylcarbodiimide (“DCC”), 4-dimethylamino pyridine and N,N-dimethylformamide to obtain a suspension and maintaining the suspension for a length of time sufficient to obtain paliperidone palmitate.

In another embodiment, the present invention encompasses paliperidone palmitate, substantially free of palmitic acid

In another embodiment, the present invention encompasses a process for preparing paliperidone palmitate substantially free of palmitic acid comprising precipitating paliperidone palmitate from a mixture of paliperidone palmitate and a solvent selected from the group consisting of: C₁ to C₅ alcohols, C₅ to C₁₂ cyclic or acyclic hydrocarbons, C₆ to C₁₂ aromatic hydrocarbons, C₂ to C₆ alkyl acetates, C₄ to C₁₀ cyclic or acyclic ethers, acetonitrile, C₂ to C₄ diols, dimethyl carbonate, diethyl carbonate, C₃ to C₆ ketones and C₃ to C₆ amides. Preferably, the obtained paliperidone palmitate is substantially free of palmitic acid.

In another embodiment, the present invention encompasses pure-paliperidone palmitate containing less than about 0.2% of any other ester with a fatty acid as determined by percentage area HPLC, and may be substantially free of palmitic acid.

In another embodiment, the present invention encompasses a process for preparing pure paliperidone palmitate comprising the steps of: a) combining paliperidone, symmetric or asymmetric palmitic anhydride, a pyridine derivative, and an organic solvent to obtain a suspension; b) maintaining the suspension for a length of time sufficient to obtain paliperidone palmitate and c) precipitating the obtained paliperidone palmitate from a mixture of paliperidone palmitate and a solvent selected from the group consisting of: C₁ to C₅ alcohols, C₅ to C₁₂ cyclic or acyclic hydrocarbons, C₆ to C₁₂ aromatic hydrocarbons, C₂ to C₆ alkyl acetates, C₄ to C₁₀ cyclic or acyclic ethers, acetonitrile, C₂ to C₄ diols, dimethyl carbonate, diethyl carbonate, C₃ to C₆ ketones and C₃ to C₆ amides to obtain pure paliperidone palmitate. Preferably, the pure paliperidone palmitate contains less than about 0.2% by HPLC of any other ester with a fatty acid and is substantially free of palmitic acid.

The present invention further encompasses 3-[2-[4-(6-fluoro-1,2-benzoisoxazol-3-yl)-1-piperidyl]ethyl]-6,7,8,9-tetrahydro-9-hydroxy-2-methyl-4H-pyrido[1,2-a]pyrimidin-4-one tetradecanoate ester (“paliperidone myristate”) and 3-[2-[4-(6-fluoro-1,2-benzoisoxazol-3-yl)-1-piperidyl]ethyl]-6,7,8,9-tetrahydro-9-hydroxy-2-methyl-4H-pyrido[1,2-a]pyrimidin-4-one octadecanoate ester (“paliperidone stearate”), and processes for their preparation thereof and their use as reference markers and reference standards to analyze the purity of paliperidone palmitate.

In another embodiment, the invention encompasses analytical methods for determining the impurity profile of paliperidone palmitate.

### BRIEF DESCRIPTION OF FIGURES

FIG. 1 illustrates the ¹H NMR spectrum of paliperidone alkoxide in the low ppm region.

FIG. 2 illustrates the ¹H NMR spectrum of paliperidone alkoxide in the high ppm region.

FIG. 3 illustrates the FAB-mass spectrum of paliperidone alkoxide.

FIG. 4 illustrates the ¹H NMR spectrum of paliperidone myristate.

FIG. 5 illustrates the ¹³C NMR spectrum of paliperidone myristate.

FIG. 6 illustrates the ¹H NMR spectrum of paliperidone stearate.

FIG. 7 illustrates the ¹³C NMR spectrum of paliperidone stearate.

### DETAILED DESCRIPTION OF THE INVENTION

As used herein, the term “room temperature” refers to a temperature of about 15° C. to about 35° C. Preferably, the room temperature may be at a temperature of about 20° C. to about 25° C.

As used herein, the term “overnight” refers to about 10 hours to about 14 hours. Preferably, overnight refers to about 12 hours.

As used herein, the term “any other ester with a fatty acid” refers to any ester of an aliphatic monocarboxilic acid, other than palmitic acid, with C₄ to C₂₈ carbon atoms.

As used herein, the term “paliperidone palmitate substantially free of palmitic acid” refers to paliperidone palmitate having less than about 0.1% by HPLC of palmitic acid.

The present invention encompasses pure paliperidone palmitate and processes for the purification of paliperidone palmitate. These processes do not require chromatographic separation, as described in US '952 or US '556 for the preparation of its derivatives, they undergo a simple recovery process and therefore are more suitable for use on an industrial scale.

The present invention encompasses paliperidone alkoxide and a process for its preparation. Paliperidone alkoxide is an intermediate in the process for preparing paliperidone palmitate. The alkoxide intermediate can be isolated prior to preparing paliperidone palmitate. Not to be limited by theory, it is believed that isolating the alkoxide intermediate from the reaction avoids the formation of undesired by-products, such as dimers obtained in an intermolecular reaction of paliperidone. Alternatively, since dimers do not form under basic conditions, the paliperidone palmitate can be prepared via a one-pot process without isolating the alkoxide intermediate. This enables one to avoid operations relating to the alkoxide separation.

Embodiments for the process of preparing paliperidone alkoxide and process for preparing paliperidone palmitate using paliperidone alkoxide as the intermediate can be summarized in the following scheme:

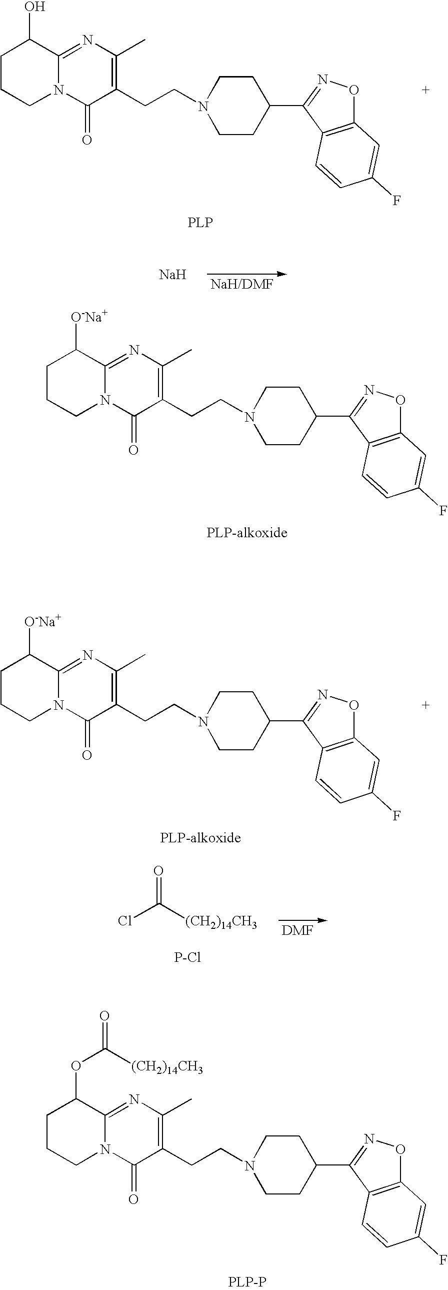

In one embodiment, the present invention encompasses paliperidone alkoxide of the following formula:

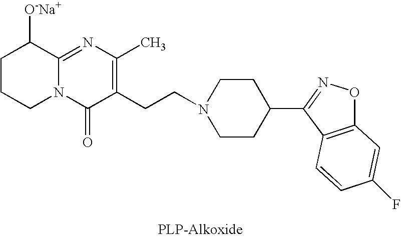

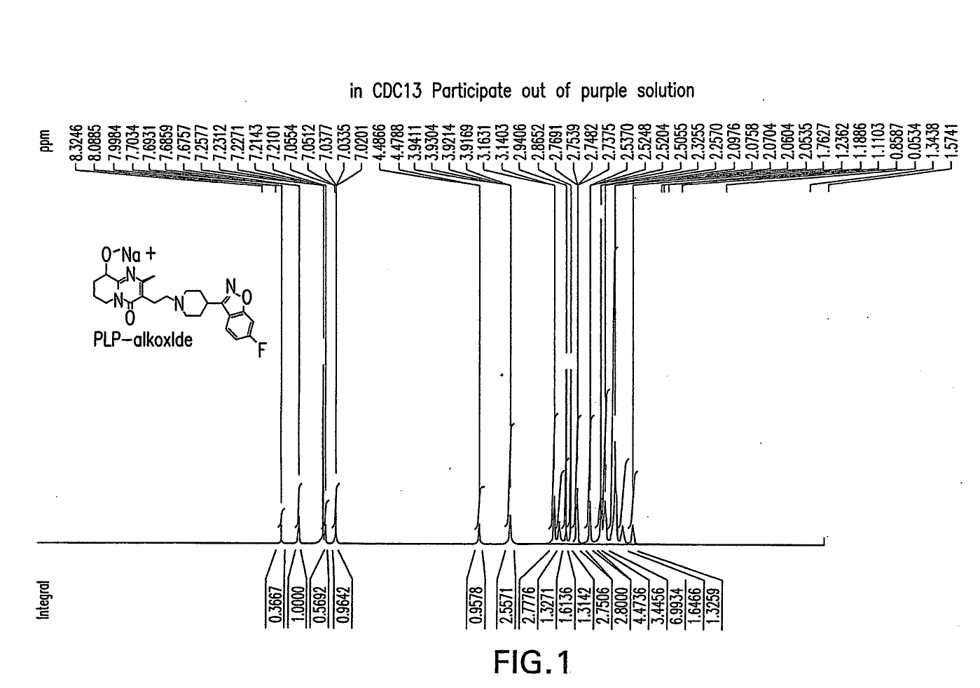

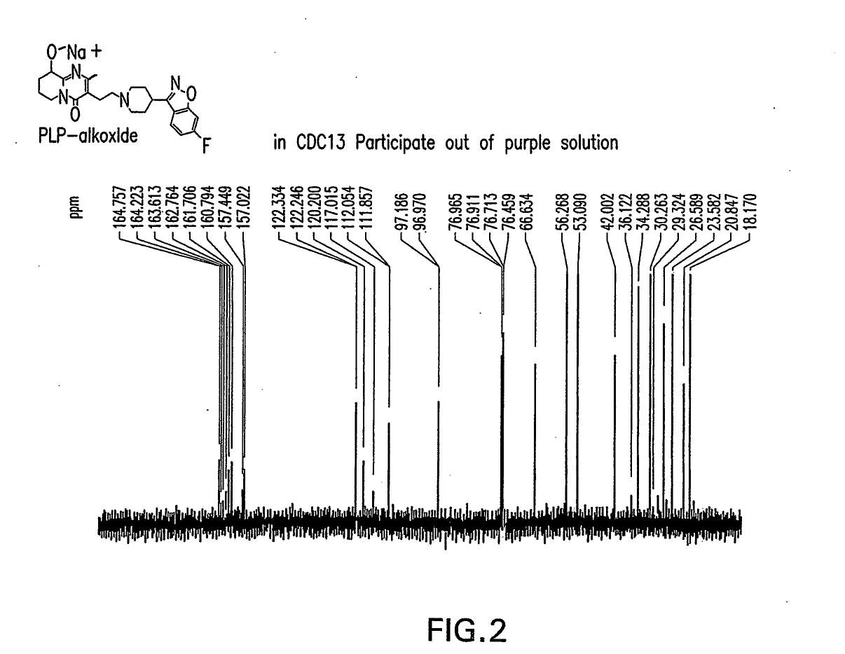

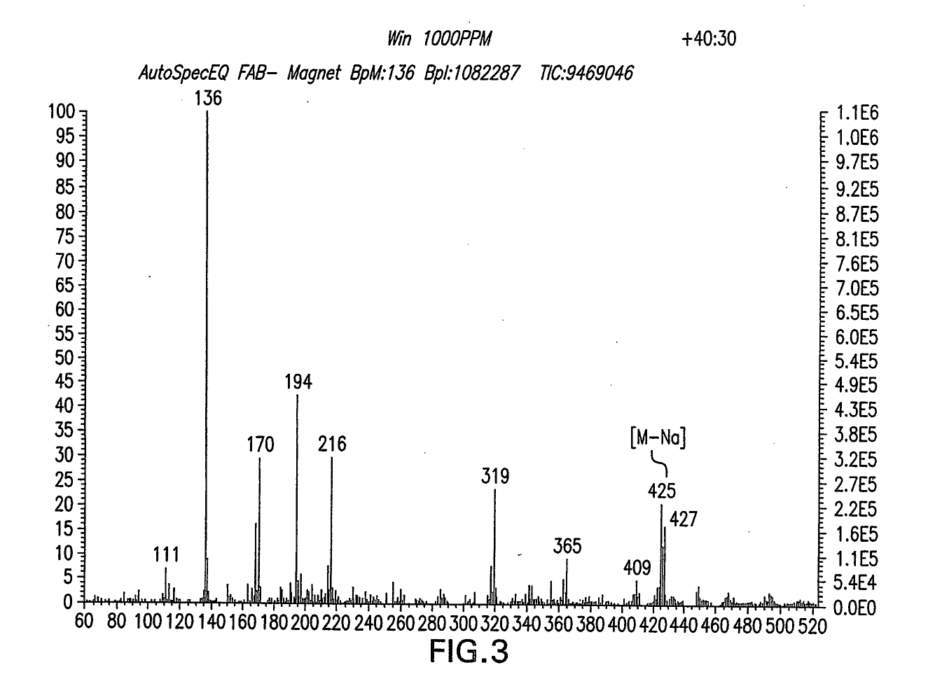

Paliperidone alkoxide can be characterized by ¹H NMR, it is observed by the absence of hydrogen corresponding to an alcohol at 5.66 ppm as depicted in FIGS. 1 and 2; and by FAB-MS showing a peak at m/e 425 [M-Na] as depicted in FIG. 3.

The invention encompasses a process for preparing paliperidone alkoxide comprising the steps of: a) combining paliperidone with at least one organic solvent to obtain a suspension; b) adding at least one base to the suspension to obtain a reaction mixture; and c) maintaining the mixture for a length of time sufficient to obtain paliperidone alkoxide.

Preferably, the length of time sufficient to obtain paliperidone alkoxide is about 2 to about 48 hours. More preferably, the length of time sufficient to obtain paliperidone alkoxide is about 15 hours to about 20 hours.

Paliperidone can be prepared according to any method known in the art, for example, according to US '952.

Preferably, the process for preparing paliperidone alkoxide may be performed under nitrogen atmosphere.

Preferably, after combining paliperidone and the organic solvent, the suspension may be maintained for about 10 minutes to 20 minutes at about room temperature. The base may then be added to the suspension slowly and a reaction mixture is obtained. The reaction mixture may be maintained for about 2 hours to about 48 hours. Preferably, the reaction mixture may be maintained for about 15 hours to about 20 hours. More preferably, it may be maintained for about 18 hours.

Preferably, the organic solvent may be selected from the group consisting of acetonitrile, dichloromethane, dimethyl sulfoxide (“DMSO”), dimethylamine (“DMA”), C₃₋₆ amides, C₃₋₆ ketones, C₆₋₁₂ aromatic hydrocarbons, C₂₋₆ alkyl acetates and C₂₋₈ ethers. Preferred C₃₋₆ amides are dimethylacetamide and dimethylformamide. Preferred C₃₋₆ ketones are acetone, methyl ethyl ketone (“MEK”) and methyl iso-butyl ketone (“MIBK”). Preferred C₆₋₁₂ aromatic hydrocarbons are benzene, toluene, o-xylene, p-xylene and m-xylene. Preferred C₂₋₆ alkyl acetates are ethyl acetate and isobutyl acetate. Preferably, the C₂₋₈ ethers are C₄₋₈ ethers. Preferred C₄₋₈ ethers are tetrahydrofuran (“THF”), diethoxymethane (“DEM”), isobutyl methyl ether, dibutyl ether, and polyethylene glycol methyl ether (“PGME”). Preferably, the organic solvent used may be acetonitrile, dichloromethane, toluene, DMF, DMA or DMSO. More preferably, the organic solvent may be dichloromethane, toluene, DMF, DMA or DMSO. Most preferably, the organic solvent may be DMF.

The base may be selected from the group consisting of alkali metal hydroxide, metal alkoxides (e.g., alkoxides of sodium, lithium or potassium) and sodium hydride (“NaH”). Preferably, the base may be sodium hydroxide, NaH or sodium tert-butoxide, and most preferably, it may be NaH. The base may also serve as a sodium source to afford a paliperidome alkoxide salt that may be isolated from the reaction.

Paliperidone and the base are present in the reaction mixture at a molar ratio that will provide the formation of paliperidone alkoxide. A person skilled in the art will know to optimize the components ratios according to the base which is used. For example, when using NaH as the base, the paliperidone and the base are present in the reaction mixture at a molar ratio of about 1:0.9 to about 1:2 of paliperidone to base. Preferably, at a molar ratio of about 1:1 to about 1:1.2, more preferably, they are present in the reaction mixture at a molar ratio of about 1:1 of paliperidone to base.

Optionally, a phase transfer catalyst (“PTC”) may be added to the paliperidone and the organic solvent. Preferably, the PTC may be selected from the group consisting of tetra alkyl ammonium halide, tetra aryl ammonium halide, tetra(alkyl)ₘ(aryl)₄₋ₘ ammonium halide, wherein m is between 1 and 3, tetra alkyl ammonium hydroxide, tetra aryl ammonium hydroxide and tetra(alkyl)(aryl) ammonium hydroxide, wherein the alkyl can be the same or different in tetra alkyl ammonium halide/hydroxide and tetra (alkyl)(aryl) ammonium halide/hydroxide, and the aryl can be the same or different in tetra aryl ammonium halide/hydroxide and tetra(alkyl)(aryl) ammonium halide/hydroxide. Preferably, the alkyl may be C₁₋₆ alkyl. Preferably, the aryl may be C₆₋₁₀ aryl. Preferably, the halide may be chloride, bromide or iodide. Preferably, the PTC may be tetra butyl ammonium hydroxide or tricaprylmethylammonium chloride. More preferably, the PTC may be tricaprylmethylammonium chloride.

Preferably, about 0.02 to about 1 molar equivalents of the PTC to paliperidone may added to the reaction mixture, more preferably, about 0.03 to about 0.5 molar equivalents of the PTC to paliperidone may be added. Most preferably, about 0.04 to 0.10 molar equivalents of the PTC to paliperidone may be added to the reaction mixture. Preferably, about 0.05 to 0.08 molar equivalents of the PTC may be added to the reaction mixture. More preferably, about 0.06 molar equivalents of the PTC may be added to the reaction mixture.

Paliperidone alkoxide may be recovered from the reaction mixture by any conventional method known in the art, for example, by filtering under reduced pressure.

In another embodiment, the present invention encompasses a process for preparing paliperidone palmitate comprising: combining paliperidone alkoxide and palmitoyl chloride to obtain a reaction mixture; and maintaining the mixture for a length of time sufficient to obtain paliperidone palmitate. The paliperidone alkoxide which is introduced into the mixture may be dissolved in an organic solvent. Optionally, the palmitoyl chloride may be dissolved in an organic solvent. The organic solvent in this process can be the same as those of the process previously described for preparing paliperidone alkoxide.

The reaction mixture may be maintained for about 30 minutes to about 30 hours. Preferably, it is maintained for about 1 hour to about 24 hours, and more preferably, it is maintained for about 2 to about 10 hours.

Paliperidone alkoxide and palmitoyl chloride can be present in the reaction mixture at a molar ratio of about 1:1 to about 2:1. Preferably, they are present in the reaction mixture at a molar ratio of about 1.5:1.

Paliperidone palmitate can be recovered from the reaction mixture by any conventional method known in the art, for example, by filtering.

Alternatively, paliperidone palmitate may be prepared by a one-pot process comprising the steps of: a) combining paliperidone, at least one organic solvent, and at least one base to obtain a reaction mixture; b) adding palmitoyl chloride to the reaction mixture; and c) maintaining the mixture for a length of time sufficient to obtain paliperidone palmitate. In this process, the paliperidone alkoxide is not isolated.

Preferably, the length of time sufficient to obtain paliperidone palmitate is about 2 to about 10 hours. More preferably, the length of time sufficient to obtain paliperidone palmitate is about 2 to about 5 hours.

Optionally, palmitoyl chloride may be dissolved in an organic solvent to form a solution. The solution of palmitoyl chloride is then added to the reaction mixture of paliperidone, the organic solvent and the base at room temperature over a period of about 10 minutes to about 1 hour. The amount of the palmitoyl chloride present in the reaction mixture may be the same as the amount described for preparing paliperidone palmitate using the alkoxide intermediate.

The base and the organic solvent in steps (a) and (b) may be the same as those of the process previously described for preparing paliperidone alkoxide. Except when the base is sodium hydroxide, the amount of the base present in the reaction mixture may be about 5 to 15 molar equivalents of the sodium hydroxide to paliperidone, preferably, about 7 to 12 molar equivalents of the sodium hydroxide to paliperidone, more preferably, about 10 molar equivalents of the sodium hydroxide to paliperidone may be added to the reaction mixture.

Optionally, a phase transfer catalyst may be added to the paliperidone, the organic solvent and the base. The PTC may be the same as those of the process previously described for preparing paliperidone alkoxide.

Optionally, when the base is sodium hydride, the reaction mixture may be cooled to a temperature of about −5° C. to about 10° C. prior to adding the solution of palmitoyl chloride. More preferably, it may be cooled to a temperature of about 0° C.

Optionally, the process further comprises a recovery step after the reaction mixture is maintained for about 30 minutes to about 30 hours. Preferably, it is maintained for about 1 hour to about 24 hours, and more preferably, it is maintained for about 2 to about 10 hours.

The recovery step may comprise adding water and the organic solvent to the reaction mixture to form a two-phase system. After the two phases are separated, water may be added to the organic phase and adjusted to pH 2 with an acid, such as hydrochloric acid. Paliperidone palmitate is isolated from the organic phase using any method known in the art, for example, by evaporating the organic phase to dryness, spray drying or lyophilization.

In one specific embodiment, sodium hydride may be added to a cooled mixture of paliperidone and DMF. Preferably, the reaction mixture may be cooled to a temperature of about −5° C. to about 10° C. More preferably, it may be cooled to a temperature of about 0° C. Palmitoyl chloride may be then added to the cooled reaction mixture to obtain paliperidone palmitate. Preferably, the reaction mixture may be maintained for about 5 hours. Optionally, the paliperidone palmitate may be recovered or isolated using a conventional method known in the art.

The present invention encompasses paliperidone palmitate containing less than about 0.2% by HPLC of any other ester with a fatty acid.

The purity of the obtained paliperidone palmitate may be measured using HPLC. HPLC analysis of paliperidone palmitate may be preferably performed using a Zorbax® SB-Phenyl (150×4.6 mm, 5μ, Part No.: 883975-912) column having an ultraviolet detector set at 238 nm. According to the maker, Agilent Technologies (Santa Clara, Calif., USA), a Zorbax® SB-Phenyl column is made by chemically bonding a sterically-protected phenethyl stationary phase to a specially prepared, ultra-highly-purified Zorbax® porous-silica microsphere with a pore size of 80 Å and a particle size of 5 μm. The sample to be analyzed may be first dissolved in 10 mL diluent (70% water to pH 2.0 with TFA: 30% acetonitrile), sonicated, further dissolved in 2 mL THF, sonicated, further dissolved in 2 mL acetonitrile, sonicated and further diluted to volume with diluent. The gradient may be prepared from a mixture of (99.5% aqueous TFA, pH 2.0:0.5% acetonitrile): acetonitrile (70:30, v/v) followed by a mixture of (99.5% aqueous TFA, pH 2.0:0.5% acetonitrile): acetonitrile (30:70, v/v) over 40 minutes.

The present invention also encompasses a process for preparing paliperidone palmitate comprising the steps of: a) combining paliperidone, symmetric or asymmetric palmitic anhydride, a pyridine derivative, and an organic solvent to obtain a suspension; and b) maintaining the suspension for a length of time sufficient to obtain paliperidone palmitate. The obtained paliperidone palmitate preferably contains less than about 0.2% of any other ester with a fatty acid as determined by percentage area HPLC.

Preferably, the length of time sufficient to obtain paliperidone palmitate is about 1 to about 48 hours. More preferably, the length of time is about 3 to about 24 hours

Preferably, the pyridine derivative may be a dialkylamino pyridine. More preferably, the pyridine derivative may be DMAP. Preferably, about 0.1 to 3 molar equivalents of the pyridine to paliperidone may be added to the suspension. Preferably, about 0.1 to 2 molar equivalents of the pyridine to paliperidone may be added to the suspension. More preferably, about 0.25 molar to 2 equivalents of the pyridine to paliperidone may be added.

The organic solvent used in this process can be the same as those of the process previously described for preparing paliperidone alkoxide.

Paliperidone palmitate may be recovered from the reaction mixture by any conventional method known in the art, for example, by filtering the mixture under reduced pressure, washing the product with the solvent and drying the product under vacuum.

As used herein, the term “symmetric palmitic anhydride” refers to an acid anhydride having the following formula: (RCO)O(OCR), wherein the R may be CH₃(CH₂)₁₄.

As used herein, the term “asymmetric palmitic anhydride” refers to an acid anhydride having the following formula: (R₁CO)O(OCR₂) or (R₁CO)O(O₂SR₃), wherein the R₁ may be CH₃(CH₂), and R₂ and R₃ may be the alkyl group of an acyl halide or sulfonic acid chloride. Preferably, R₂ may be CH₃(CH₂)ₙ, wherein n=0-28, or Ph(CH₂)ₘ, wherein m=1-5. Preferably, R₃ may be (CH₂)ₘCH₃, (CH₂)ₘPh or Ph (CH₂)ₘCH₃, wherein m=0-5.

Preferably, the acyl halide is benzoyl chloride or pivaloyl chloride. Preferably, the sulfonic acid chloride is methane sulphonyl chloride.

The palmitic anhydride can be either purchased (e.g. from Sigma-Aldrich) or prepared in situ during the process of paliperidone palmitate preparation.

For example, the commercially available symmetric palmitic anhydride can be used for the preparation of paliperidone palmitate as is exemplified in the following scheme:

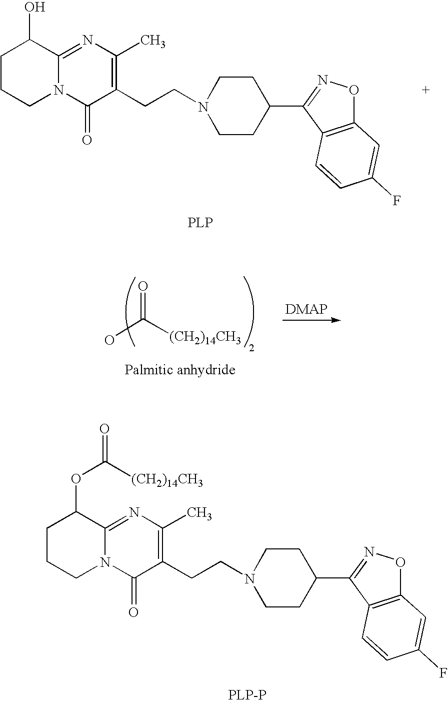

After obtaining the suspension in step (a) of the process, the suspension may be heated to about 50° C. to 80° C. and maintained for about 2 hours to about 4 hours. Preferably, the suspension may be heated to about 70° C. and maintained for about 2 hours. The suspension may then be cooled to room temperature and further maintained for about 1 hour to about 36 hours. Preferably, the suspension is maintained for about 1 hour to about 30 hours, more preferably, it is maintained for about 2 hours to about 24 hours to obtain paliperidone palmitate. Preferably, the suspension may be further maintained for about 5 hours to about 18 hours. More preferably, it may be further maintained for about 5 hours. Methyl tert-butyl ether may be added to the mixture prior to the cooling.

Preferably, the molar ratio of paliperidone to symmetric palmitic anhydride present in the suspension may be at about 1:0.5 to about 1:3, preferably, the molar ratio of paliperidone to symmetric palmitic anhydride present may be at about 1:0.8 to 1:2. Preferably, the molar ratio of paliperidone to palmitic anhydride present in the suspension may be at about 1:0.9 to about 1:1.2. More preferably, the molar ratio of paliperidone to palmitic anhydride present in the suspension may be at about 1:1.

In one specific embodiment, the invention encompasses a process for preparing paliperidone palmitate comprising: combining paliperidone, symmetric palmitic anhydride, DMAP, and an organic solvent to obtain a suspension; and maintaining the suspension for a length of time sufficient to obtain paliperidone palmitate. Preferably, the length of time sufficient to obtain paliperidone palmitate is about 5 to about 24, more preferably, the length of time is about 5 to about 18 hours.

Optionally, the asymmetric palmitic anhydride may be prepared in situ according to the Yamaguchi esterification which is exemplified in the following scheme:

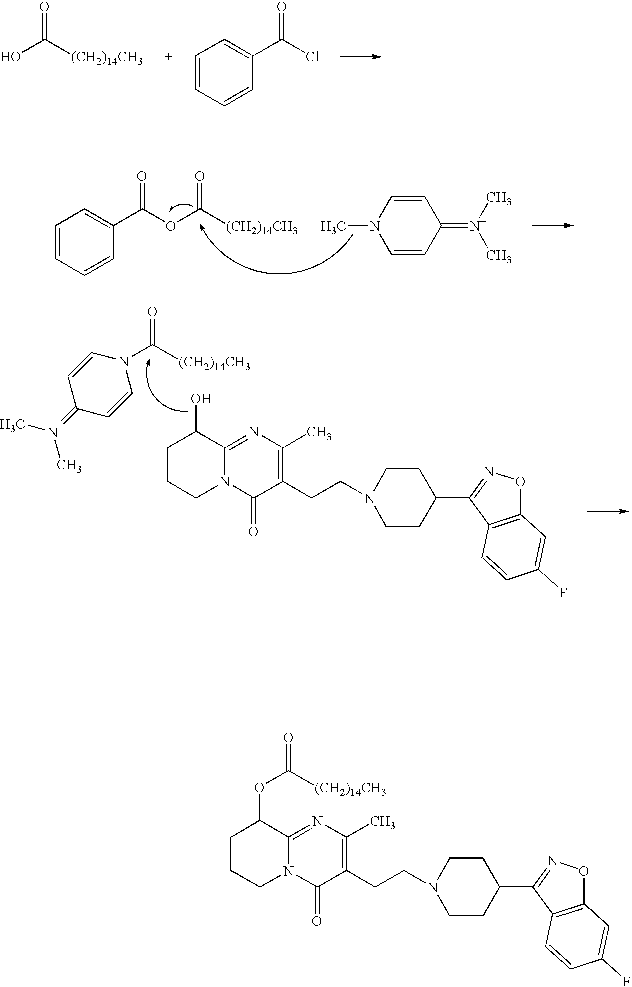

(I. Dhimitnika, J. Santa Lucia, Org. Lett., 2006, 8, 47-50, hereby incorporated by reference).

Optionally, the palmitic anhydride can be prepared in situ by reacting palmitic acid with acyl halides selected from the group consisting of R₁COX and R₂SOX, wherein R₁ is CH₃(CH₂)ₙ or Ph(CH₂)ₘ; R₂ is O(CH₂)ₘCH₃ or Ph(CH₂)ₘCH₃; and X is a halide. Preferably, R₁ is CH₃(CH₂)ₙ, wherein n=0-28, or Ph(CH₂)ₘ, wherein m=1-5; and R₂ is O(CH₂)ₘCH₃, (CH₂)ⱼPh or Ph(CH₂)ₘCH₃ wherein m—0-5. Preferably, the acyl halides are selected from the group consisting of: acetyl chloride, pivaloyl chloride, benzoyl chloride and thionyl chloride.

Preferably, the palmitic acid and the acid chloride are present in the reaction at a molar ratio of about 0.5:1 to about 1:1.5. Preferably, the molar ratio of palmitic acid to acid chloride may be at about 0.6:1 to about 0.9:1.2. More preferably, the molar ratio of palmitic acid to acid chloride present in the reaction may be at about 1:1.

Optionally, triethylamine may be added to the reaction mixture.

Preferably, after obtaining the reaction mixture in step (a), the mixture may be maintained for about 10 minutes to 20 minutes at room temperature. The acid chloride may be then added to the reaction mixture in a drop-wise fashion and the mixture may be maintained for about 5 hours to about 48 hours to obtain paliperidone palmitate. Preferably, the mixture may be maintained for about 3 hours to about 24 hours.

In one specific embodiment, the invention encompasses a process for preparing paliperidone palmitate comprising: a) combining paliperidone, palmitic acid, DMAP and an organic solvent to obtain a reaction mixture; b) adding an acid chloride selected from the group consisting of acetyl chloride, pivaloyl chloride, benzoyl chloride and thionyl chloride to the reaction mixture, and c) maintaining the mixture for a length of time sufficient to obtain paliperidone palmitate. In this process, the palmitic anhydride is prepared in situ. Preferably, the length of time sufficient to obtain paliperidone palmitate is about 2 to about 48 hours. Preferably, the mixture may be maintained for about 2 hours to about 24 hours.

Palmitic acid can often be contaminated with other fatty acids. Optionally, the palmitic acid may be purified first before adding it to the reaction mixture. Preferably, the palmitic acid after the purification process contains less than about 0.2% of any other fatty acids, e.g., palmitic acid free of any other fatty acids, as determined by percentage area HPLC and consequently, the obtained paliperidone palmitate contains less than about 0.2% of any other ester with a fatty acid as determined by percentage area HPLC.

The present invention further encompasses a process for purifying palmitic acid comprising the steps of: a) dissolving palmitic acid in a solvent selected from the group consisting of: saturated or unsaturated, linear or branched, cyclic or acyclic C₅ to C₈ hydrocarbons, C₃ to C₆ ketones, C₆ to C₁₂ aromatic hydrocarbons, C₂ to C₆ alkyl acetates and acetonitrile to obtain a suspension; and b) maintaining the suspension for a length of time sufficient to obtain crystalline palmitic acid.

Preferably, the length of time sufficient to obtain crystalline palmitic acid may be about 2 hours to about 48 hours.

Preferred saturated or unsaturated, linear or branched, cyclic or acyclic C₅ to C₈ hydrocarbons are cyclohexane, hexane, octane and heptane. Preferred C₃₋₆ ketones are acetone, methyl ethyl ketone and methyl iso-butyl ketone. Preferred C₆₋₁₂ aromatic hydrocarbons are benzene, toluene, o-xylene, p-xylene and m-xylene. Preferred C₂₋₆ alkyl acetates are ethyl acetate and isobutyl acetate. Preferably the solvent is cyclohexane, toluene, hexane, octane or heptane. More preferably, the solvent is heptane.

Preferably, a solution of palmitic acid and the solvent may be combined at the reflux temperature of the solvent to complete dissolution, and further cooled to facilitate crystallization. Preferably, the suspension may be cooled to a temperature of about 20° C. to 24° C. More preferably, the suspension may be cooled to a temperature of about 22° C. The suspension is then preferably maintained for about 1 hr.

The palmitic acid may be further re-crystallized, as described above in order to remove substantially all other impurities.

Preferably, about 5 to 10 molar equivalents of the solvent to paliperidone may be added to the suspension containing palmitic acid. Preferably, about 7 to 9 molar equivalents of the solvent to paliperidone may be added to the suspension. More preferably, about 8.5 molar equivalents of the solvent to paliperidone may be added to the suspension.

In a most preferred embodiment, the present invention encompasses a process for preparing paliperidone palmitate comprising the steps of: crystallizing palmitic acid from heptane; combining the crystalline palmitic acid thus obtained with paliperidone, DMAP, an organic solvent and an acid chloride selected from the group consisting of: acetyl chloride, pivaloyl chloride, benzoyl chloride and thionyl chloride to obtain a reaction mixture; and maintaining the mixture for a length of time sufficient to obtain paliperidone palmitate. The obtained paliperidone palmitate preferably contains less than about 0.2% of any other ester with a fatty acid as determined by percentage area HPLC.

Preferably, the length of time sufficient to obtain paliperidone palmitate is about 2 to about 48 hours.

The present invention further encompasses a process for preparing paliperidone palmitate in the presence of DCC and a catalytic amount of DMAP. This process can be summarized in the following scheme:

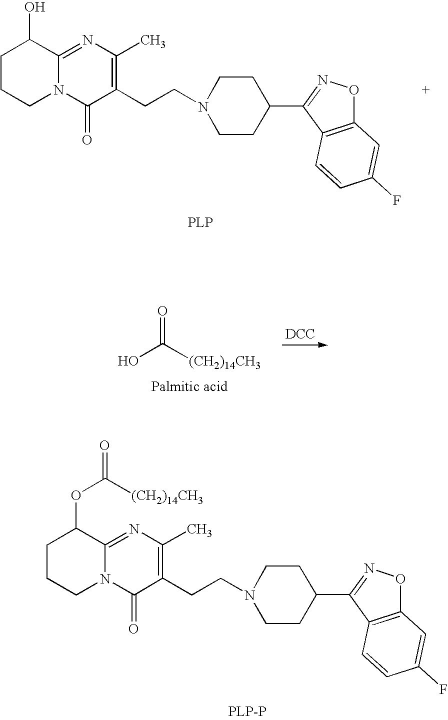

In one embodiment, the present invention encompasses a process for preparing paliperidone palmitate comprising the steps of: combining paliperidone, palmitic acid, N,N′-dicyclohexylcarbodiimide, 4-dimethylamino pyridine and N,N-dimethylformamide to obtain a suspension and maintaining the suspension for a length of time sufficient to obtain paliperidone palmitate.

Preferably, the length of time sufficient to obtain paliperidone palmitate is about 2 hours to about 24 hours.

Preferably, the molar ratio of the paliperidone to DCC present in the suspension may be at about 1:0.9 to about 1:2. Preferably, they are present in the suspension at a molar ratio of about 1:1 to about 1:1.2. More preferably, the molar ratio of paliperidone to DCC may be at about 1:1

Preferably, about 0.1 to 0.5 molar equivalents of the DMAP to paliperidone may be added to the suspension. Preferably, about 0.1 to 0.3 molar equivalents of the DMAP to paliperidone may be added to the suspension. More preferably, about 0.2 molar equivalents of the DMAP to paliperidone may be added.

Paliperidone palmitate can be recovered from the suspension by any conventional method known in the art, for example, by filtering the obtained product, washing the product with the solvent and drying the product under vacuum.

The present invention further encompasses paliperidone palmitate, substantially free of palmitic acid and processes for preparing paliperidone palmitate, substantially free of palmitic acid. The paliperidone palmitate substantially free of palmitic acid can be characterized by a DSC thermogram showing an endothermic peak at about 117-118° C.

The purity of the obtained paliperidone palmitate (from any other ester with a fatty acid and/or from palmitic acid) may be measured using HPLC. HPLC analysis of paliperidone palmitate may be preferably performed using a Zorbax® SB-Phenyl (250×4.6 mm, 5μ, Part No.: 880975-912) column having a Corona®^{plus} CAD® Detector. According to the maker, Agilent Technologies (Santa Clara, Calif., USA), a Zorbax® SB-Phenyl column is made by chemically bonding a sterically-protected phenethyl stationary phase to a specially prepared, ultra-highly-purified Zorbax® porous-silica microsphere with a pore size of 80 Å and a particle size of 5 μm. The sample to be analyzed may be first dissolved in 4 mL THF. The column is eluted with isocratic mixture of aqueous TFA, pH 2.0 and acetonitrile (50:50, v/v) followed by a gradient elution with acetonitriole.

The present invention encompasses a process for preparing paliperidone palmitate substantially free of palmitic acid comprising precipitating paliperidone palmitate from a mixture of paliperidone palmitate and a solvent selected from the group consisting of: C₁ to C₅ alcohols, C₅ to C₁₂ cyclic or acyclic hydrocarbons, C₆ to C₁₂ aromatic hydrocarbons, C₂ to C₆ alkyl acetates, C₄ to C₁₀ cyclic or acyclic ethers, acetonitrile, C₂ to C₄ diols, dimethyl carbonate, diethyl carbonate, C₃ to C₆ ketones and C₃ to C₆ amides. Preferably, the obtained paliperidone palmitate is substantially free of palmitic acid.

Preferably the C₁ to C₅ alcohols are methanol, ethanol, n-propanol, isopropyl alcohol, n-butanol, and n-pentanol. Preferably, the C₅ to C₁₂ cyclic or acyclic hydrocarbons are cyclohexane, hexane, octane and heptane. Preferably, the C₆ to C₁₂ aromatic hydrocarbons are benzene, toluene, o-xylene, p-xylene and m-xylene. Preferably, the C₂₋₆ alkyl acetates are ethyl acetate and isobutyl acetate. Preferably, the C₄ to C₁₀ cyclic or acyclic ethers are diethyl ether, methyl tert-butyl ether (“MTBE”), and THF. Preferably, the C₂ to C₄ diols are ethylene glycol and diethylene glycol (“DEG”). Preferably, the C₃₋₆ ketones are acetone, methyl ethyl ketone and methyl iso-butyl ketone. Preferably, the C₃₋₆ amides are dimethylacetamide and dimethylformamide. Preferably the solvent may be toluene, methyl tert-butyl ether or ethanol.

Preferably, a mixture of paliperidone palmitate and the solvent may be combined at the reflux temperature of the solvent to complete dissolution, and further cooled to facilitate crystallization. Preferably, the suspension may be cooled to room temperature.

In a specific embodiment, the present invention encompasses a process for preparing paliperidone palmitate substantially free of palmitic acid comprising slurrying paliperidone palmitate in MTBE. Preferably, a suspension of paliperidone palmitate in MTBE may be maintained overnight at room temperature.

Yet in another specific embodiment, the present invention encompasses a process for preparing paliperidone palmitate substantially free of palmitic acid comprising crystallizing paliperidone palmitate from ethanol. Preferably, a suspension of the paliperidone palmitate and the ethanol may be obtained at the reflux temperature of the solvent, and cooled to facilitate crystallization.

Preferably, the suspension may be cooled to a temperature of about 20° C. to 24° C. More preferably, the suspension may be cooled to a temperature of about 22° C. The suspension may be then preferably maintained for about 1 hr.

Preferably, in the processes for preparing paliperidone palmitate substantially free of palmitic acid, the amount of the solvent may be present at about 10 ml to 30 ml per gram of paliperidone palmitate. More preferably, the amount of the solvent may be present at about 15 ml to 25 ml per gram of paliperidone palmitate.

Preferably, in the processes for preparing paliperidone palmitate substantially free of palmitic acid, paliperidone palmitate may be recovered from the suspension or mixture by any conventional method known in the art, for example, by filtering and drying.

The present invention further encompasses pure paliperidone palmitate containing less than about 0.2% of any other ester with a fatty acid as determined by percentage area HPLC, and may be substantially free of palmitic acid.

The present invention also encompasses a process for preparing pure paliperidone palmitate comprising the steps of: a) combining paliperidone, symmetric or asymmetric palmitic anhydride, a pyridine derivative, and an organic solvent to obtain a suspension; b) maintaining the suspension for a length of time sufficient to obtain paliperidone palmitate and c) precipitating the obtained paliperidone palmitate from a mixture of paliperidone palmitate and a solvent selected from the group consisting of: C₁ to C₅ alcohols, C₅ to C₁₂ cyclic or acyclic hydrocarbons, C₆ to C₁₂ aromatic hydrocarbons, C₂ to C₆ alkyl acetates, C₄ to C₁₀ cyclic or acyclic ethers, acetonitrile, C₂ to C₄ diols, dimethyl carbonate, diethyl carbonate, C₃ to C₆ ketones and C₃ to C₆ amides. Preferably, the precipitated paliperidone palmitate contains less than about 0.2% by HPLC of any other ester with a fatty acid and is substantially free of palmitic acid.

The present invention further encompasses 3-[2-[4-(6-fluoro-1,2-benzoisoxazol-3-yl)-1-piperidyl]ethyl]-6,7,8,9-tetrahydro-9-hydroxy-2-methyl-4H-pyrido[1,2-a]pyrimidin-4-one tetradecanoate ester (“paliperidone myristate”) represented by the following formula:

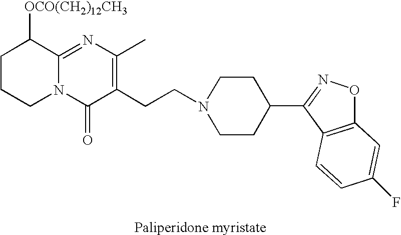

and 3-[2-[4-(6-fluoro-1,2-benzoisoxazol-3-yl)-1-piperidyl]ethyl]-6,7,8,9-tetrahydro-9-hydroxy-2-methyl-4H-pyrido[1,2-a]pyrimidin-4-one octadecanoate ester (“paliperidone stearate”) represented by the following formula:

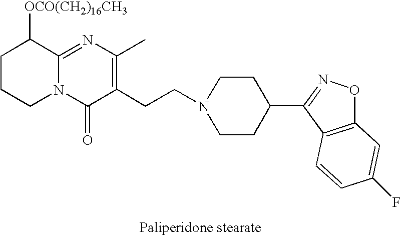

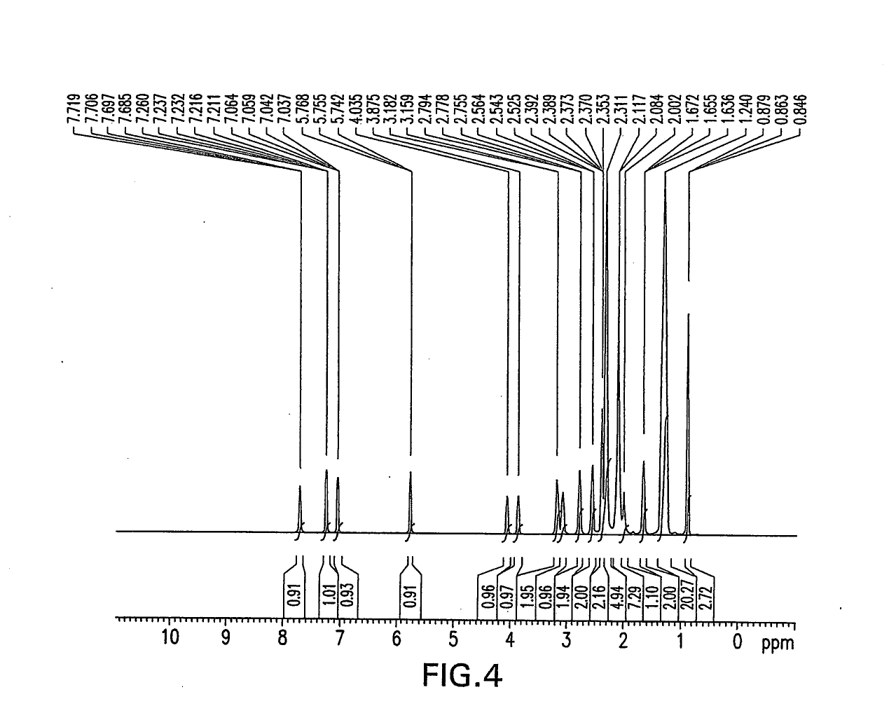

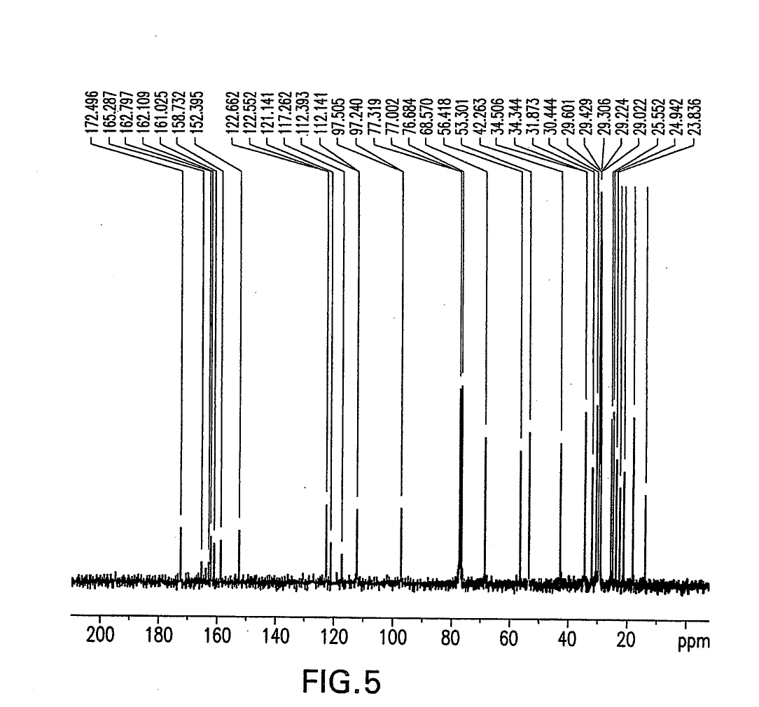

The present invention further encompasses isolated paliperidone myristate characterized by ¹H NMR as depicted in FIG. 4 and by ¹³C NMR as depicted in FIG. 5.

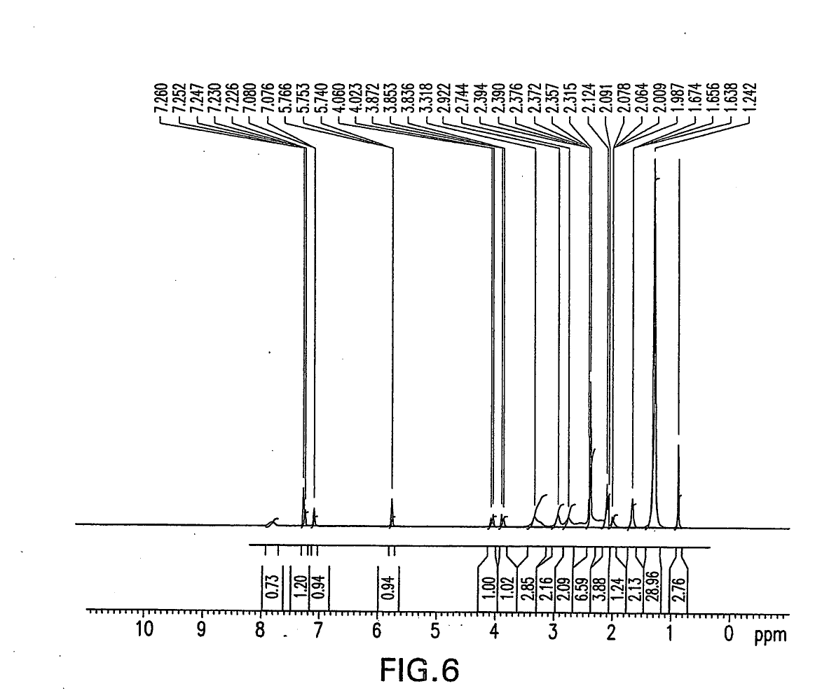

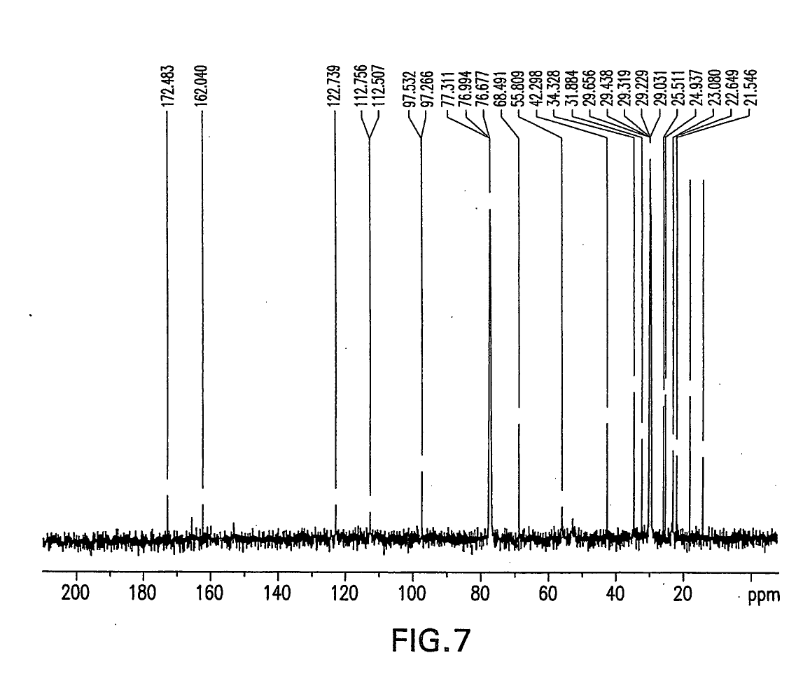

The present invention further encompasses isolated paliperidone stearate characterized by ¹H NMR as depicted in FIG. 6 and by ¹³C NMR as depicted in FIG. 7.

The present invention encompasses a process for preparing paliperidone myristate (or stearate) comprising: combining paliperidone, symmetric or asymmetric myristic (or stearic) anhydride, a pyridine derivative, and an organic solvent to obtain a reaction mixture; and maintaining the mixture for a length of time sufficient to obtain paliperidone myristate (or stearate).

Preferably, the length of time sufficient to obtain paliperidone myristate (or stearate) is about 3 hours to about 24 hours.

As used herein, the term “symmetric myristic (or stearic) anhydride” refers to an acid anhydride having the following formula: (RCO)O(OCR), wherein the R may be CH₃(CH₂)₁₂ [or CH₃(CH₂)₁₆₁].

As used herein, the term “asymmetric myristic (or stearic) anhydride” refers to an acid anhydride having the following formula: (R₁CO)O(OCR₂) or (R₁CO)O(O₂SR₃), wherein the R₁ may be CH₃(CH₂)₁₂ (or CH₃(CH₂)₁₆) and R₂ may be the alkyl group of an acyl halide or sulfonic acid chloride and wherein when the “asymmetric myristic (or stearic) anhydride” is an acid anhydride having the formula of (R₁ CO)O(OCR₂), R₁ and R₂ are not the same.

Preferably, the myristic (or stearic) anhydride may be prepared in situ using myristic (or stearic) acid and an acyl chloride as detailed above for the preparation of asymmetric palmitic anhydride.

The reaction conditions and ratios in the process for preparing paliperidone myristate (or stearate) can be the same as those of the process previously described for the preparation of paliperidone palmitate using palmitic anhydride.

As used herein, the term “reference standard” refers to a compound that may be used both for quantitative and qualitative analysis of an active pharmaceutical ingredient. For example, the HPLC retention time of the reference standard compound allows a relative retention time with respect to the active pharmaceutical ingredient to be determined, thus making qualitative analysis possible. Furthermore, the concentration of the compound in solution before injection into an HPLC column allows the areas under the HPLC peaks to be compared, thus making quantitative analysis possible.

A “reference marker” is used in qualitative analysis to identify components of a mixture based upon their position, e.g., in a chromatogram or on a Thin Layer Chromatography (TLC) plate (Strobel, pp. 921, 922, 953). For this purpose, the compound does not necessarily have to be added to the mixture if it is present in the mixture. A “reference marker” is used only for qualitative analysis, while a reference standard may be used for quantitative or qualitative analysis, or both. Hence, a reference marker is a subset of a reference standard, and is included within the definition of a reference standard.

The paliperidone myristate and paliperidone stearate of the present invention are useful as a reference markers for paliperidone palmitate. As such, they may be used in order to detect the paliperidone myristate and/or paliperidone stearate impurity in a paliperidone palmitate sample. The present invention also encompasses a method for detecting the paliperidone myristate and/or paliperidone stearate impurity in a paliperidone palmitate sample comprising: a) providing a reference sample containing paliperidone palmitate and paliperidone myristate or paliperidone stearate; b) performing HPLC analysis on the reference sample to determine the relative retention time of paliperidone palmitate compared to paliperidone myristate or paliperidone stearate; c) performing HPLC analysis on the paliperidone palmitate sample to determine the relative retention time of paliperidone palmitate compared to paliperidone myristate or paliperidone stearate; and d) comparing the relative retention times determined in steps b) and c); wherein, if the relative retention times determined in steps b) and c) are the same, then paliperidone myristate or paliperidone stearate are identified as being the same as the reference marker.

The paliperidone myristate or paliperidone stearate of the present invention can also be used as reference standards for paliperidone palmitate. Paliperidone myristate or paliperidone stearate may be used to quantify impurities in a paliperidone palmitate sample. A sample of paliperidone palmitate may be spiked with a known amount of paliperidone myristate or paliperidone stearate and analyzed by HPLC to identify peaks associated with the impurities. Impurity levels in paliperidone palmitate can be determined by comparing the area percent of the impurities by HPLC with the area percent of the paliperidone myristate or paliperidone stearate injected in a known amount within linearity ranges. A control sample without added paliperidone myristate or paliperidone stearate can be run to determine the area percent of the paliperidone myristate or paliperidone stearate peak associated with the added amount of paliperidone myristate or paliperidone stearate. Alternatively, at least two samples can be prepared by adding known and differing amounts of paliperidone myristate or paliperidone stearate to the samples. The proportion of the HPLC peak due to the paliperidone palmitate present in the mixture without the addition of paliperidone myristate or paliperidone stearate can be determined by plotting the HPLC peak area against the amount of paliperidone myristate or paliperidone stearate added to each of the samples, and extrapolating the plot to zero.

The present invention further encompasses a method of determining the amount of the paliperidone myristate or paliperidone stearate impurity in a paliperidone palmitate sample comprising: a) measuring by HPLC the area under a peak corresponding to paliperidone myristate or paliperidone stearate in a reference standard comprising a known amount of paliperidone myristate or paliperidone stearate; b) measuring by HPLC the area under a peak corresponding to paliperidone myristate or paliperidone stearate in a paliperidone palmitate sample containing paliperidone myristate or paliperidone stearate; and c) determining the amount of paliperidone myristate or paliperidone stearate in the paliperidone palmitate sample by comparing the area of step a) to the area of step b).

The present invention encompasses an HPLC method for the determination of the impurity profile of paliperidone palmitate.

Having thus described the invention with reference to particular preferred embodiments and illustrative examples, those in the art can appreciate modifications to the invention as described and illustrated that do not depart from the spirit and scope of the invention as disclosed in the specification. The examples are set forth to aid in understanding the invention but are not intended to, and should not be construed to, limit its scope in any way. Absent statement to the contrary, any combination of the specific embodiments described above are consistent with and encompassed by the present invention.

### EXAMPLES

### Example 1

### Preparation of Paliperidone Palmitate Using Dichloromethane and Tetra-Butyl Ammonium Hydroxide

A 250 ml three-neck flask with a magnetic stirrer and dropping funnel was charged with 5 g of paliperidone (11.7 mmol, 1 eq), 105 ml dichloromethane, 2 ml of tetra butyl ammonium hydroxide (7 mmol, 0.6 eq), and 10 ml (115 mmol, 10 eq) of a solution of sodium hydroxide (22%). A solution of palmitoyl chloride (3.9 ml, 13 mmol, 1 eq, in 10 ml dichloromethane) was added over 10 minutes and stirred at room temperature. After 5 hours, 50 ml of water and 50 ml of methanol were added and the resulting phases were separated. After phase separation, water was added to the organic phase and adjusted to pH 2 with HCl. The organic phase was evaporated to dryness resulting in the crude paliperidone palmitate

### Example 2

### Preparation of Paliperidone Palmitate Using Toluene with Tricaprylmethylammonium Chloride

A 250 ml three neck flask with a magnetic stirrer and dropping funnel was charged with 5 g of paliperidone (11.7 mmol, 1 eq), 50 ml toluene and 0.25 ml (0.546 mmol, 0.05 eq) of tricaprylmethylammonium chloride followed by 10 ml (115 mmol, 10 eq) of a solution of sodium hydroxide (22%). A solution of palmitoyl chloride (3.9 ml, 13 mmol, 1 eq, in 10 ml toluene) was added over 1 hour and stirred at room temperature for an additional hour. After 5 hours, 50 ml of water and 50 ml of methanol were added and the resulting phases were separated. After phase separation, water was added to the organic phase and adjusted to pH 2 with HCl. The organic phase was evaporated to dryness resulting in the crude paliperidone palmitate.

### Example 3

### Preparation of Paliperidone Palmitate Using NaH

A 100 ml three neck flask equipped with a magnetic stirrer was charged with 75 ml of N,N-dimethylformamide and sodium hydride 60% (0.47 g, 11.7 mmol, 1 eq). The suspension was stirred at 22±2° C. for 10 minutes; and 5 g of paliperidone (11.7 mmol, 1 eq) were added in portions to obtain a dark clear solution. After 1 hour stirring, 3.94 ml of palmitoyl chloride (12.9 mmol, 1.1 eq) were added drop-wise to obtain a slurry and further stirred at the same temperature for 2 hours. The reaction mixture was put into 150 ml water and the obtained heavy precipitate, was stirred for 30 minutes, filtered under reduced pressure, rinsed with water and dried at 55° C. under reduced pressure for overnight.

### Example 4

### Preparation of Paliperidone Palmitate Using NaH and at Low Temperature

A 100 ml reactor equipped with a mechanical stirrer was charged with 75 ml of N,N-dimethylformamide and 5 g paliperidone (11.7 mmol, 1 eq) under nitrogen. The mixture was cooled to 0° C. and 0.58 g (14.5 mmol, 1.2 eq) of sodium hydride (60%) was added slowly. The suspension was stirred at 0° C. and 4 ml (13 mmol, 1 eq) of palmitoyl chloride were added drop-wise and the mixture was kept at the same temperature for an additional 5 hours. The reaction mixture was put into 75 ml water and the obtained heavy precipitate, was stirred at room temperature, filtered and washed with water (2×20 ml). The solid was dried at 50° C. under reduced pressure for overnight resulting in 6.9 g of a solid. Yield: 53.1 by wt.

### Example 5

### Preparation of Paliperidone Palmitate Using Sodium Tert-Butoxide

A 100 ml 3-neck flask equipped with a magnetic stirrer was charged with 75 ml of toluene, 5 g of paliperidone (11.7 mmol, 1 eq) and 1.24 g (12.9 mmol, 1 eq) of sodium tert-butoxide, and the suspension stirred at 22±2° C. After 15 minutes, 3.94 ml (13 mmol, 1 eq) of palmitoyl chloride were added drop-wise slowly and the resulting mixture was stirred at the same temperature for 24 hours.

### Example 6

### Preparation of Paliperidone Palmitate by Isolation of Paliperidone Alkoxide

a) Preparation of Paliperidone Alkoxide

A 50 ml flask equipped with a magnetic stirrer was charged under nitrogen atmosphere with paliperidone (2 g, 4.69 mmol, 1 eq) and N,N-dimethylformamide (30 ml), and the suspension was stirred at room temperature for 10 minutes. Sodium hydride (0.18 g, 4.69 mmol, 1 eq) was added slowly, the reaction mixture was further stirred for 18 hours, filtered under reduced pressure and nitrogen atmosphere to obtain paliperidone alkoxide as a yellow solid.

b) Preparation of Paliperidone Palmitate from Paliperidone Alkoxide

To a mixture of 1.85 g of paliperidone alkoxide (obtained according to example 6a) in 30 ml DMF was added 1.85 ml of palmitoyl chloride and stirred for 2 hours at room temperature. The resulting solid was filtrated to give paliperidone palmitate. Yield: 10% by HPLC.

### Example 7

### Purification of Paliperidone Palmitate by Slurrying

A 50 ml flask equipped with a magnetic stirrer was charged with 20 ml of MTBE and 1 g of paliperidone palmitate and stirred at room temperature for overnight. The mixture was filtrated and dried in a vacuum oven at 50° C. resulting in paliperidone palmitate free of palmitic acid.

### Example 8

### Crystallization of Paliperidone Palmitate

A 100 ml flask equipped with a magnetic stirrer was charged with 90 ml of IPA and 6 g of paliperidone palmitate. The mixture was heated to reflux till dissolution, and cooled to room temperature. The resulting solid was filtrated, washed with 6 ml IPA and dried in a vacuum oven at 50° C. resulting in 4.67 g of paliperidone palmitate. Yield: 77.8% by wt.

### Example 9

### Preparation of Paliperidone Palmitate in the Presence of DCC

A 20 ml vial equipped with a magnetic stirrer was charged with paliperidone (0.5 g, 1.17 mmol, 1 eq), palmitic acid (0.3 g, 1.17 mmol, 1 eq), N,N′-dicyclohexylcarbodiimide (0.27 g, 1.31 mmol, 1 eq), dimethylamino pyridine (0.015 g, 0.122 mmol, 0.10 eq) and N,N-dimethylformamide (5 ml). The suspension was stirred overnight at room temperature. Deionized water (10 ml) was then added and stirred for 30 minutes, filtered under reduced pressure, rinsed with water and dried at 55° C. under vacuum. Yield: 39.2% by weight.

### Example 10

### Preparation of Paliperidone Palmitate Using Palmitic Anhydride

A 20 ml vial equipped with a magnetic stirrer was charged with paliperidone (0.5 g, 1.17 mmol, 1 eq), palmitic anhydride (0.58 g, 1.17 mmol, 1 eq), 4-dimethylamino pyridine (0.03 g, 0.245 mmol, 0.2 eq) and toluene (5 ml). The suspension was heated to 70° C. and was stirred for 2 hours, then cooled to room temperature and was left stirring overnight. Then, methyl t-butyl ether (5 ml) was added and the suspension was further stirred overnight at room temperature, filtered under reduced pressure, rinsed with methyl t-butyl ether (0.5 ml) and dried at 50° C. under vacuum. The resulting solid was paliperidone palmitate. Yield: 46.2% by weight.

### Example 11

### Preparation of Paliperidone Palmitate Using Benzoyl Chloride

A 100 ml flask equipped with a magnetic stirrer was charged with paliperidone (5 g, 11.7 mmol, 1 eq), palmitic acid (3 g, 11.7 mmol, 1 eq), 4-dimethylaminopyridine (0.36 g, 2.95 mmol, 0.25 eq), triethylamine (3.25 ml, 23.3 mmol, 2 eq) and tetrahydrofuran (50 ml). The suspension was stirred at room temperature for 15 minutes. Benzoyl chloride (0.95 ml, 8.17 mmol, 0.7 eq) was added drop-wise and the suspension was further stirred at room temperature for 3-24 hours. Water (50 ml) was then added and the suspension was further stirred for 1 hour then filtered off under vacuum, washed with water and dried at 50° C. under vacuum. The resulting solid was paliperidone palmitate (purity: 95.0% area by HPLC, yield: 6.25 g, 80.0%).

### Example 12

### Preparation of Paliperidone Palmitate Using Pivaloyl Chloride

A 100 ml flask equipped with a magnetic stirrer was charged with 5 g of paliperidone (11.7 mmol, 1 eq), 3 g of palmitic acid, 0.36 g (2.95 mmol, 0.25 eq) of 4-dimethylaminopyridine, 3.25 ml (23.3 mmol, 2 eq) of triethylamine and 50 ml of tetrahydrofuran. The suspension was stirred at room temperature for 15 minutes and 1.44 ml (7.96 mmol, 0.7 eq) pivaloyl chloride was added drop-wise and the suspension was further stirred at room temperature for 24 hours. After this time 50 ml of water were added and the suspension was further stirred for 1 hour, filtered off under vacuum, washed with water and dried at 50° C. under vacuum. The resulting solid was paliperidone palmitate (purity: 97.5% area by HPLC, yield: 6.25 g, 80.0%).

### Example 13

### Preparation of Paliperidone Palmitate Using Acetyl Chloride

A 100 ml flask equipped with a magnetic stirrer was charged with 5 g of paliperidone (11.7 mmol, 1 eq), 3 g of palmitic acid, 0.36 g (2.95 mmol, 0.25 eq) of 4-dimethylaminopyridine, 3.25 ml (23.3 mmol, 2 eq) of triethylamine and 50 ml of tetrahydrofuran. The suspension was stirred at room temperature for 15 minutes and 0.70 ml (9.89 mmol, 0.8 eq) acetyl chloride was added drop-wise and the suspension was further stirred at room temperature for 24 hours. After this time 50 ml of water were added and the suspension was further stirred for 1 hour, filtered off under vacuum, washed with water and dried at 50° C. under vacuum. The resulting solid was paliperidone palmitate (purity: 29.2% area by HPLC).

### Example 14

### Preparation of Paliperidone Palmitate Using Thionyl Chloride

A 100 ml flask equipped with a magnetic stirrer was charged with 5 g of paliperidone (11.7 mmol, 1 eq), 6 g of palmitic acid, 0.36 g (2.95 mmol, 0.25 eq) of 4-dimethylaminopyridine, 3.25 ml (23.3 mmol, 2 eq) of triethylamine and 50 ml of tetrahydrofuran. The suspension was stirred at room temperature for 15 minutes and 0.85 ml (11.65 mmol, 1 eq) thionyl chloride was added drop-wise and the suspension was further stirred at room temperature for 24 hours. After this time 50 ml of water were added and the suspension was further stirred for 1 hour, filtered off under vacuum, washed with water and dried at 50° C. under vacuum. The resulting solid was paliperidone palmitate (purity: 44.1% area by HPLC).

### Example 15

### Preparation of Paliperidone Palmitate Using Palmitic Anhydride

A 250 ml reactor equipped with a mechanical stirrer and a reflux condenser was charged under N₂ with paliperidone (10 g, 23.45 mmol, 1 eq), palmitic anhydride (14.2 g, 28.7 mmol, 1.2 eq), 4-dimethylaminopyridine (0.72 g, 5.9 mmol, 0.25 eq) and toluene (100 ml). The suspension was heated to 70° C. and was stirred for 2 hours, then cooled to 22°±2° C. The suspension was stirred at the same temperature for 1 hour. methyl t-butyl ether (100 ml) was added and the suspension was cooled to 10° C. and was stirred for 1 hour at the same temperature. The suspension was then filtered under reduced pressure, rinsed with methyl t-butyl ether (40 ml) and dried at 50° C. under vacuum. The resulting solid was paliperidone palmitate crude (yield: 29.35 g, purity: 99.7% area by HPLC, compounds related to palmitic acid were less then 0.15%). Yield: 88.5% by wt.

### Example 16

### Purification of Palmitic Acid

A 500 ml reactor equipped with a mechanical stirrer and a reflux condenser was charged with palmitic acid (100.0 g, 0.39 mol) and heptane (500 ml, 3.4 mol, 8.5 eq). The suspension was stirred and heated to reflux temperature to complete dissolution then was cooled to 22°±2° C. The suspension was stirred at the same temperature for 1 hour and was filtered off under reduced pressure. The wet solid was recrystallized in the same way as above, collected by filtration under reduced pressure and was dried at 50° C. under reduced pressure to obtain pure palmitic acid. Yield: 64.7% by wt.

### Example 17

### Purification of Paliperidone Palmitate with Ethanol

A 250 ml reactor equipped with a mechanical stirrer and a reflux condenser was charged with paliperidone palmitate crude (29.0 g) and ethanol absolute (435 ml). The suspension was stirred and heated to reflux temperature to complete dissolution then was cooled off to 22°±2° C. The suspension was stirred at the same temperature for 1 hour, filtered under reduced pressure, rinsed with ethanol absolute and dried at 50° C. under vacuum over night. The resulting solid was pure paliperidone palmitate (purity: 99.8% area by HPLC, other fatty acid esters mainly myristate and stearate were less then 0.1%). Yield: 90.0% by wt.

### Example 18

### Preparation of Paliperidone Myristate

A 100 ml flask equipped with a magnetic stirrer was charged with paliperidone (5 g, 11.7 mmol, 1 eq), myristic acid (2.67 g, 11.7 mmol, 1 eq), 4-dimethylaminopyridine (0.36 g, 2.95 mmol, 0.25 eq), triethylamine (3.25 ml, 23.3 mmol, 2 eq) and tetrahydrofuran (50 ml). The suspension was stirred at room temperature for 15 minutes. Benzoyl chloride (1.55 ml, 13.34 mmol, 1.1 eq) was added drop-wise and the suspension was further stirred at room temperature for 6 hours. Water (50 ml) was then added and the suspension was further stirred for 1 hour then filtered off under vacuum, washed with water and dried at 50° C. under vacuum. The resulting solid was paliperidone myristate (purity: 97.5% area by HPLC, yield: 6.25 g, 95%).

### Example 19

### Preparation of Paliperidone Stearate

A 100 ml flask equipped with a magnetic stirrer is charged with paliperidone (5 g, 11.7 mmol, 1 eq), stearic acid (3.33 g, 11.7 mmol, 1 eq), 4-dimethylaminopyridine (0.36 g, 2.95 mmol, 0.25 eq), triethylamine (3.25 ml, 23.3 mmol, 2 eq) and tetrahydrofuran (50 ml). The suspension is stirred at room temperature for 15 minutes. Benzoyl chloride (1.55 ml, 13.34 mmol, 1.1 eq) is added drop-wise and the suspension is further stirred at room temperature for 24 hours. Water (50 ml) is then added and the suspension is further stirred for 1 hour then filtered off under vacuum, washed with water and dried at 50° C. under vacuum. The resulting solid is paliperidone stearate. Yield: 96% by wt.

### Example 20

### Determining the Impurity Profile of Paliperidone Palmitate by HPLC

The product was analyzed by HPLC using Zorbax® SB-Phenyl (150×4.6 mm, 5μ, Part No.: 883975-912) as the column. The mobile phase was a mixture of solutions A and B as a gradient shown in the table below:

| Time (min) | Solution A (%) | Solution B (%) |
|---|---|---|
| 0 | 70 | 30 |
| 40 | 30 | 70 |
| 50 | 30 | 70 |

Solution A was prepared from 99.5% H₂O to pH 2.0 with TFA: 0.5% acetonitrile. Solution B was prepared from acetonitrile. The flow-rate was 1.3 ml/min and UV detector was set to 238 nm.

### Example 21

### Determining the Content of Palmitic Acid in Paliperidone Palmitate by HPLC

The product was analyzed by HPLC using Zorbax® SB-Phenyl (250×4.6 mm, 5μ, Part No.: 880975-912) as the column. The mobile phase was a mixture of solutions A and B as a gradient shown in the table below:

| Time (min) | Solution A (%) | Solution B (%) |
|---|---|---|
| 0 | 50 | 50 |
| 30 | 50 | 50 |
| 30.1 | 0 | 100 |
| 40 | 0 | 100 |

Solution A was prepared from H₂O to pH 2.0 with TFA. Solution B was prepared from acetonitrile. The flow-rate was 1.4 ml/min and the detector used was Corona®^{Plus} CAD® Detector.

### Example 22

### DSC Measurement

3-5 mg of material was weighed into an aluminum crucible that was sealed and pierced 3 times. The crucible was heated between the range of 25 to 250° C. at a rate of 10° C. per min. The DSC furnace was purged with 40 ml per min of N₂.

1. A paliperidone palmitate containing less than about 0.2% by HPLC of any other ester with a fatty acid as determined by percentage area HPLC.

2. A process for preparing paliperidone palmitate comprising the steps of:
a) combining paliperidone, symmetric or asymmetric palmitic anhydride, a pyridine derivative, and an organic solvent to obtain a suspension; and
b) maintaining the suspension for a sufficient time to obtain paliperidone palmitate.

3. The process of claim 2, wherein the obtained paliperidone palmitate contains less than about 0.2% by HPLC of any other ester with a fatty acid as determined by percentage area HPLC.

4. The process of claim 2, wherein the pyridine derivative is a dialkylamino pyridine.

5. The process of claim 4, wherein the pyridine derivative is a dimethylamino pyridine.

6. The process of claim 2, wherein the organic solvent is selected from the group consisting of acetonitrile, dichloromethane, dimethyl sulfoxide, C₃₋₆ amides, C₃₋₆ ketones, C₆₋₁₂ aromatic hydrocarbons, C₂₋₆ alkyl acetates and C₄₋₈ ethers.

7. The process of claim 6, wherein the organic solvent is toluene.

8. The process of claim 2, wherein the mixture is maintained for about 1 hour to about 48 hours.

9. The process of claim 8, wherein the mixture is maintained for about 3 to about 24 hours.

10. The process of claim 2, wherein the palmitic anhydride is prepared in-situ by a process of reacting palmitic acid with acyl halides selected from the group consisting of R₁COX and R₂SOX, wherein R₁ is CH₃(CH₂)ₙ, with n=0-28, or Ph(CH₂)ₘ, with m=1-5; R₂ is O(CH₂)ₘCH₃, (CH₂)ₘPh or Ph(CH₂)ₘCH₃, with m=0-5; and X is a halide.

11. The process of claim 10, wherein the acyl halides are selected from the group consisting of: acetyl chloride, pivaloyl chloride, benzoyl chloride and thionyl chloride.

12. A process for purifying palmitic acid comprising the steps of:
a) dissolving palmitic acid in a solvent selected from the group consisting of: saturated or unsaturated, linear or branched, cyclic or acyclic C₅ to C₈ hydrocarbons, C₃ to C₆ ketones, C₆ to C₁₂ aromatic hydrocarbons, C₂ to C₆ alkyl acetates and acetonitrile to obtain a suspension; and
b) maintaining the suspension to obtain crystalline palmitic acid,
wherein the obtained palmitic acid contains less than 0.2% by HPLC of any other ester with a fatty acid.

13. The process of claim 12, wherein the solvent is cyclohexane, toluene, hexane, octane or heptane.

14. The process of claim 12, wherein the process further comprises heating the suspension at the reflux temperature of the solvent and cooling the suspension to a temperature of about 20° C. to 24° C.

15. The process of claim 12, wherein the mixture is maintained for about 2 to about 48 hours.

16. A paliperidone palmitate containing less than 0.1% by HPLC of palmitic acid.

17. A process for preparing paliperidone palmitate containing less than 0.1% by HPLC of palmitic acid comprising precipitating paliperidone palmitate from a mixture of paliperidone palmitate and a solvent selected from the group consisting of C₁ to C₅ alcohols, C₅ to C₁₂ cyclic or acyclic hydrocarbons, C₆ to C₁₂ aromatic hydrocarbons, C₂ to C₆ alkyl acetates, C₄ to CIO cyclic or acyclic ethers, acetonitrile, C₂ to C₄ diols, dimethyl carbonate, diethyl carbonate, C₃ to C₆ ketones and C₃ to C₆ amides to obtain paliperidone palmitate containing less than 0.1% by HPLC of palmitic acid.

18. The process of claim 17, wherein the solvent is toluene, methyl tert-butyl ether or ethanol.

19. The process of claim 17, wherein the process further comprises heating the suspension at the reflux temperature of the solvent and cooling the suspension to a temperature of about room temperature.

20. The paliperidone palmitate of claim 1 further having less than about 0.1% by HPLC of palmitic acid.

21. A process for preparing paliperidone palmitate comprising the steps of:
a) crystallizing palmitic acid from a solvent selected from the group consisting of: saturated or unsaturated, linear or branched, cyclic or acyclic C₅ to C₈ hydrocarbons, C₃ to C₆ ketones, C₆ to C₁₂ aromatic hydrocarbons, C₂ to C₆ alkyl acetates and acetonitrile to obtain palmitic acid containing less than 0.2% by HPLC of any other ester with a fatty acid;
b) combining palmitic acid containing less than 0.2% by HPLC of any other ester with a fatty acid with paliperidone, 4-dimethylamino pyridine, an organic solvent and an acyl halide selected from the group consisting of R₁COX and R₂SOX, wherein R₁ is CH₃(CH₂)ₙ, with n=0-28, or Ph(CH₂)ₘ, with m=1-5; R₂ is O(CH₂)ₘCH₃, (CH₂)ₘPh or Ph(CH₂)ₘCH₃, with m=0-5; and X is a halide; and
c) maintaining the mixture for about 1 to about 48 hours to obtain paliperidone palmitate,
wherein the obtained paliperidone palmitate preferably contains less than about 0.2% of any other ester with a fatty acid as determined by percentage area HPLC.

22. The process of claim 21 further comprising precipitating paliperidone palmitate from a mixture of paliperidone palmitate and a solvent selected from the group consisting of C₁ to C₅ alcohols, C₅ to C₁₂ cyclic or acyclic hydrocarbons, C₆ to C₁₂ aromatic hydrocarbons, C₂ to C₆ alkyl acetates, C₄ to C₁₀ cyclic or acyclic ethers, acetonitrile, C₂ to C₄ diols, dimethyl carbonate, diethyl carbonate, C₃ to C₆ ketones and C₃ to C₆ amides to obtain paliperidone palmitate having less than about 0.2% by HPLC of any other ester with a fatty acid and less than about 0.1% by HPLC of palmitic acid.

23. A process for preparing paliperidone palmitate comprising the steps of:
a) crystallizing palmitic acid from heptane to obtain palmitic acid containing less than 0.2% by HPLC of any other ester with a fatty acid;
b) combining palmitic acid containing less than 0.2% by HPLC of any other ester with a fatty acid with paliperidone, 4-dimethylamino pyridine, an organic solvent and an acid chloride selected from the group consisting of: acetyl chloride, pivaloyl chloride, benzoyl chloride and thionyl chloride to obtain a reaction mixture; and
c) maintaining the mixture for about 1 to about 48 hours to obtain paliperidone palmitate,
wherein the obtained paliperidone palmitate preferably contains less than about 0.2% of any other ester with a fatty acid as determined by percentage area HPLC.

24. The process of claim 23 further comprising precipitating paliperidone palmitate from a mixture of paliperidone palmitate and a solvent selected from the group consisting of C₁ to C₅ alcohols, C₅ to C₁₂ cyclic or acyclic hydrocarbons, C₆ to C₁₂ aromatic hydrocarbons, C₂ to C₆ alkyl acetates, C₄ to C₁₀ cyclic or acyclic ethers, acetonitrile, C₂ to C₄ diols, dimethyl carbonate, diethyl carbonate, C₃ to C₆ ketones and C₃ to C₆ amides to obtain paliperidone palmitate having less than about 0.2% by HPLC of any other ester with a fatty acid and less than about 0.1% by HPLC of palmitic acid.

25-69. (canceled)
